(19) United States
(12) Patent Application Publication
(10) Pub. No.: US 2008/0242717 A1
(43) Pub. Date: Oct. 2, 2008

(54) METHODS FOR TREATING BENIGN PROSTATIC HYPERPLASIA

(76) Inventors: Fumiyasu SATO, Azumino City (JP); Yoshio Furihata, Tokyo (JP)

Correspondence Address: KENYON & KENYON LLP, ONE BROADWAY, NEW YORK, NY, 10004, US

(21) Appl. No.: 12/038,598

(22) Filed: Feb. 27, 2008

Related U.S. Application Data
(60) Provisional application No. 60/892,170, filed on Feb. 28, 2007.

(51) Int. Cl. A61K 31/405; A61P 19/02

(52) U.S. Cl. 514/415

(57) ABSTRACT

Patients having symptoms associated with benign prostatic hyperplasia can be treated with a once daily composition containing silodosin or a pharmaceutically acceptable salt thereof.

### CROSS-REFERENCE TO RELATED APPLICATIONS

This application claims the benefit of U.S. Provisional Application Ser. No. 60/892,170, filed Feb. 28, 2007, hereby incorporated by reference.

### FIELD OF THE INVENTION

The invention relates to compositions and methods for treating patients having lower urinary tract symptoms associated with benign prostatic hyperplasia with silodosin or a pharmaceutically acceptable salt thereof.

### BACKGROUND OF THE INVENTION

Silodosin is an indoline derivative having the molecular formula C₂₅H₃₂F₃N₃O₄ and the following chemical structure.

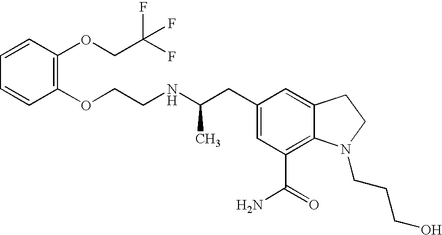

Silodosin is an α-adrenergic antagonist that has high selectivity for the α_{1A} receptor relative to α_{1B} and α_{1D} receptors. Silodosin is approved in Japan for 2 and 4 mg twice daily dosing to treat symptoms associated with benign prostatic hyperplasia (“BPH”). The synthesis of silodosin is described in U.S. Pat. No. 5,387,603, which is incorporated herein by reference in its entirety.

BPH is non-cancerous growth of the prostate gland, which most typically occurs in middle-aged and elderly men. The symptoms of BPH include, for example, weak or intermittent urinary stream (flow rate), straining when urinating, a hesitation before urine flow starts, a sense that the bladder has not emptied completely, and dribbling at the end of urination or leakage afterward.

The American Urological Association has developed the International Prostate Symptom Score (“IPSS”) as a way to measure the severity of voiding symptoms associated with BPH. To calculate the IPSS, the patient ranks the frequency of the following symptoms on a scale of 0 to 5 (with 0 being “not at all” and 5 being “almost always”): sensation of incomplete emptying of the bladder after urination; frequent urination; intermittent urination; difficulty in postponing urination; weak urinary stream; straining to begin urination; frequency of urination at night. The scores for each symptom are added together to get the total IPSS. The higher the IPSS score, the more severe the voiding symptoms. A score of 0-7 is considered to be mildly symptomatic, a score of 8-19 is considered to be moderately symptomatic, and a score of 20-35 is considered to be severely symptomatic. The IPSS is also referred to as the American Urological Association Symptom Index Score (“AUASS”).

Silodosin has been found to be effective in treating the symptoms associated with BPH. A phase III randomized, placebo-controlled, double blind study has shown that administering 4 mg of silodosin to a patient twice daily has comparable efficacy in treating LUTS associated with benign prostatic hyperplasia to that achieved when administering 0.2 mg of tamsulosin (currently marketed as FLOMAX®) to a patient once daily. See Yoshida, M., “Silodosin, A New Effective α_{1A}-Adrenoceptor Selective Antagonist for the Treatment of Benign Prostatic Hyperplasia: Results of a Phase 3 Randomized, Placebo-Controlled, Double-Blind Study,” J. Urol., 173(4 Suppl.): 1-467, Abstract 1642 (April 2005), which is incorporated herein by reference in its entirety. The pharmacokinetics of silodosin are described, for example in the following articles: Matsubara, Y., et al., “Pharmacokinetics and Disposition of Silodosin (KMD-3213),” Yakugaku Zasshi, 126, 237-245 (2006) and Shimizu, T., et al. “Pharmacokinetic Profile of Silodosin in Clinical Practice,” Yakugaku Zasshi, 126, 257-263 (2006), both of which are incorporated herein in their entireties by reference.

Once-daily dosing regimens are generally preferred over twice-daily dosing regimens because the former is generally more convenient and increases patient compliance. Accordingly, there is a need in the art for additional methods for treating the symptoms associated with BPH, particularly ones that include once-daily administration with silodosin.

### SUMMARY OF THE INVENTION

In one embodiment, the invention encompasses a method for treating a patient having symptoms associated with BPH comprising administering once daily to said patient a pharmaceutical composition comprising an effective amount of silodosin or a pharmaceutically acceptable salt thereof.

The invention also encompasses a method for achieving comparable efficacy in treating a patient having symptoms associated with BPH, comprising administering to said patient once daily a pharmaceutical composition comprising twice the dose of silodosin, or a pharmaceutically acceptable salt thereof, that is administered in each dose in a twice daily regimen, wherein there is no concomitant increase in cardiovascular adverse side effects relative to that observed with the twice daily regimen of a silodosin.

In another embodiment, the invention encompasses a method for treating a patient having symptoms associated with benign prostatic hyperplasia over a 24-hour period, comprising administering once daily to said patient a pharmaceutical composition comprising an effective amount of silodosin or a pharmaceutically acceptable salt thereof, wherein silodosin plasma levels of about 25%, or less, of Cₘₐₓ at steady state are achieved in said patient at 12 hours after administration. Preferably, the silodosin plasma level at about 12 hours is about 20%, or less, of Cₘₐₓ at steady state, and more preferably the silodosin plasma level at about 12 hours is about 15%, or less, of Cₘₐₓ at steady state.

In another embodiment, the invention encompasses a pharmaceutical composition comprising more than 4 mg, preferably about 8 mg, of silodosin or a pharmaceutically acceptable salt thereof and a pharmaceutically acceptable excipient in a unitary dosage form.

Preferred pharmaceutical compositions for use in the once-daily dosing regimens of the invention comprise an immediate release dosage form. In addition, preferred embodiments contain about 8 mg silodosin or a pharmaceutically acceptable salt thereof in a unitary dosage.

### BRIEF DESCRIPTION OF THE DRAWINGS

FIG. 1 illustrates blood plasma concentrations (ng/ml) observed over a 24-hour period after the administration of a 4 mg dose of silodosin once daily, a 8 mg dose of silodosin once daily, and a simulated 4 mg dose of silodosin twice daily (from Tables 1a, b, and c).

FIG. 2 illustrates blood plasma concentrations (ng/ml) observed over a 24-hour period after the administration of a 8 mg dose of silodosin once daily and a simulated 4 mg dose of silodosin twice daily (from Tables 2b and c).

FIG. 3 illustrates the change from baseline (CFB) in IPSS/AUASS during silodosin and placebo therapy in study A over a 4-week period.

FIG. 4 illustrates the change from baseline (CFB) in IPSS/AUASS during silodosin and placebo therapy in study B over a 12-week period.

FIG. 5 illustrates the change from baseline (CFB) in IPSS/AUASS during silodosin and placebo therapy in study D over a 12-week period.

FIG. 6 illustrates the change from baseline (CFB) in IPSS/AUASS during silodosin and placebo therapy in study E over a 12-week period.

FIG. 7 illustrates the change from baseline (CFB) in maximum flow rate (Qₘₐₓ) (ml/sec) during silodosin and placebo therapy in study A over a 4-week period.

FIG. 8 illustrates the change from baseline (CFB) in maximum flow rate (Qₘₐₓ) (ml/sec) during silodosin and placebo therapy in study B over a 12-week period.

FIG. 9 illustrates the change from baseline (CFB) in maximum flow rate (Qₘₐₓ) (ml/sec) during silodosin and placebo therapy in study D over a 12-week period.

FIG. 10 illustrates the change from baseline (CFB) in maximum flow rate (Qₘₐₓ) (ml/sec) during silodosin and placebo therapy in study E over a 12-week period.

### DETAILED DESCRIPTION OF THE INVENTION

A patient having symptoms associated with BPH may be treated by administering a once-daily regimen of a pharmaceutical composition containing an effective amount of silodosin or a pharmaceutically acceptable salt thereof.

As used herein, when two compositions are stated to have “comparable efficacy,” it means that the clinical responses (such as IPSS) seen either within a single study, or between different studies, are of similar magnitude. Preferably, the IPSS produced by two compositions having comparable efficacy differs by less than about 2 and/or the Qₘₐₓ produced by two compositions having comparable efficacy differs by less than about 0.5 ml/min after 4 to 8 weeks of treatment.

As used herein “effective amount” means an amount of silodosin or a pharmaceutically acceptable salt thereof that provides relief of BHP symptoms in a patient with BPH. Measures used to determine relief of BPH symptoms include, for example, maximum flow rate (“Qₘₐₓ”) and IPSS/AUASS. In preferred embodiments, the effective amount of silodosin is more than about 4 mg, about 4 mg to about 8 mg, about 4 mg, or about 8 mg.

As used herein “pharmaceutically acceptable salt” means those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 1977, 66: 1 et seq. The salts may be prepared in situ during the final isolation and purification of the compounds of the invention or separately by reacting a free base function with a suitable acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable basic addition salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

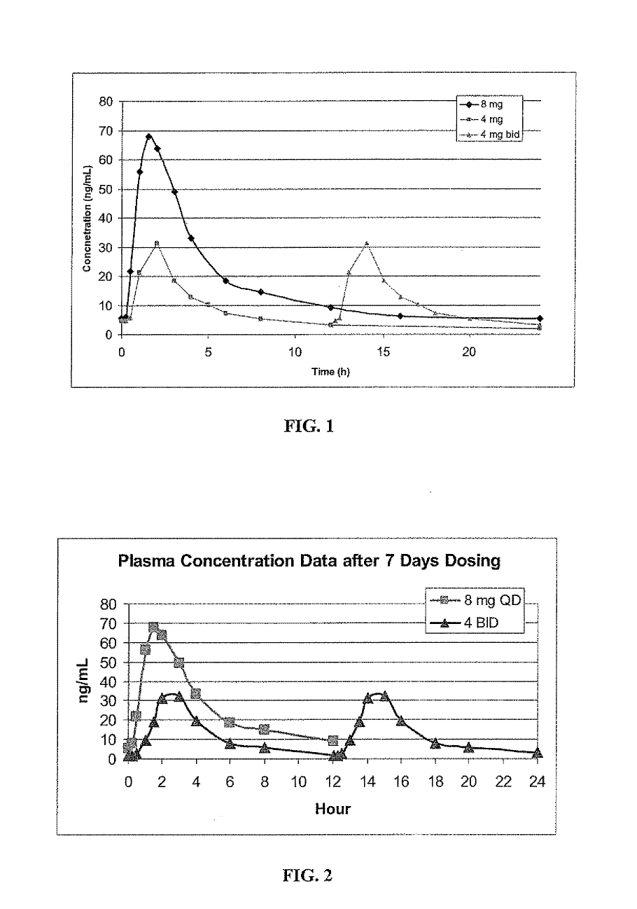

Preferably, the pharmaceutical composition of silodosin is administered in an immediate-release dosage form. As used herein “immediate release” means any dosage form that is adapted to release about 50% or more, preferably about 60% or more, more preferably about 75% or more, of the active drug from the dosage form one hour from administration of said dosage form. When a preferred immediate-release dosage form of silodosin is administered to a patient once daily, the maximum blood plasma concentration of silodosin (Cₘₐₓ) is reached at about 2 hours after administration at steady state conditions and falls to near baseline about 12 hours after administration. This is illustrated in FIGS. 1 and 2 and Tables 1a-c and 2a-c. The data in Table 1a is derived from study 98364, the protocol for which is described in Example 2 below. The data in Tables 1b and 2a-b is derived from study US011, the protocol for which is described in Example 1 below. The data in Tables 1c and 2c is simulated based upon the data in Tables 1a and 2a, respectively, assuming that there is no accumulation between doses. Steady state conditions can be determined by one of ordinary skill in the art and exist, for example, by six days of once-daily administration of a preferred immediate-release dosage form of silodosin.

TABLE 1a
Blood Plasma Concentrations (ng/ml) Observed on Day 7 with
a 4 mg Dose of Silodosin Once Daily for 7 Days

| | Time (hours) | | | | | | | | | | | |
|---|---|---|---|---|---|---|---|---|---|---|---|---|
| Subject | 0 (t₀) | 0.25 | 0.5 | 1 | 2 | 3 | 4 | 5 | 6 | 8 | 12 | 24 |
| 1 | 6.36 | 7.06 | 11.82 | 31.27 | 39.99 | 24.30 | 15.36 | 12.59 | 8.60 | 6.13 | 4.01 | 2.98 |
| 2 | 2.77 | 2.76 | 3.49 | 13.96 | 20.57 | 12.39 | 8.36 | 7.29 | 4.72 | 4.58 | 2.09 | 1.15 |
| 3 | 3.35 | 3.35 | 2.93 | 15.35 | 23.58 | 14.33 | 11.22 | 7.42 | 5.52 | 3.83 | 1.44 | 0.50 |
| 4 | 4.64 | 4.89 | 4.51 | 15.95 | 35.34 | 24.44 | 16.60 | 13.32 | 8.90 | 6.14 | 4.54 | 2.02 |
| 5 | 6.03 | 5.28 | 5.10 | 29.55 | 37.80 | 16.81 | 13.36 | 11.12 | 8.57 | 6.58 | 4.46 | 3.38 |
| Mean | 4.63 | 4.67 | 5.57 | 21.22 | 31.46 | 18.45 | 12.98 | 10.35 | 7.26 | 5.45 | 3.31 | 2.01 |

TABLE 1b
Blood Plasma Concentrations (ng/ml) Observed on Day 7
with an 8 mg Dose of Silodosin Once Daily for 7 Days

| | Time (hours) | | | | | | | | | | | | |
|---|---|---|---|---|---|---|---|---|---|---|---|---|---|
| Subject | 0 (t₀) | 0.25 | 0.5 | 1 | 1.5 | 2 | 3 | 4 | 6 | 8 | 12 | 16 | 24 |
| 1 | 1.95 | 1.60 | 11.63 | 41.51 | 65.51 | 54.34 | 52.40 | 30.19 | 11.08 | 8.40 | 5.04 | 3.81 | 3.42 |
| 2 | 6.99 | 9.65 | 14.52 | 24.58 | 32.58 | 45.40 | 51.86 | 35.37 | 18.88 | 15.18 | 9.45 | 6.12 | 6.89 |
| 3 | 2.40 | 2.44 | 25.23 | 72.25 | 63.27 | 56.03 | 33.38 | 15.51 | 9.47 | 7.79 | 3.86 | 2.33 | 1.72 |
| 4 | 1.90 | 2.00 | 2.01 | 12.58 | 35.49 | 42.13 | 35.86 | 17.49 | 10.20 | 9.68 | 4.49 | 2.74 | 1.91 |
| 5 | 4.15 | 9.25 | 40.90 | 46.27 | 39.01 | 30.68 | 17.49 | 10.60 | 8.31 | 7.43 | 4.80 | 3.93 | 3.91 |
| 6 | 3.94 | 3.98 | 21.98 | 62.96 | 55.57 | 62.78 | 47.15 | 30.50 | 11.39 | 9.77 | 8.22 | 4.72 | 3.88 |
| 7 | 4.87 | 3.64 | 5.69 | 26.49 | 63.87 | 62.92 | 53.17 | 36.46 | 19.22 | 14.50 | 7.91 | 5.28 | 4.38 |
| 8 | 15.77 | 13.11 | 16.93 | 113.90 | 129.20 | 106.10 | 88.84 | 72.43 | 57.30 | 42.35 | 23.11 | 14.85 | 11.65 |
| 9 | 8.04 | 7.98 | 56.28 | 103.20 | 127.00 | 113.80 | 61.25 | 50.08 | 20.36 | 16.57 | 16.51 | 12.73 | 9.92 |
| Mean | 5.56 | 5.96 | 21.69 | 55.97 | 67.94 | 63.80 | 49.04 | 33.18 | 18.47 | 14.63 | 9.27 | 6.28 | 5.30 |

TABLE 1c
Estimated Blood Plasma Concentrations (ng/ml) on Day 7
with a 4 mg Dose of Silodosin Twice Daily for 7 Days
Time (hours)

| 0 (t₀) | 0.25 | 0.5 | 1 | 2 | 3 | 4 | 5 | 6 | 8 | 12 | 12.25 | 12.5 | 13 | 14 | 15 | 16 | 17 | 18 | 20 | 24 |
|---|---|---|---|---|---|---|---|---|---|---|---|---|---|---|---|---|---|---|---|---|
| 4.63 | 4.67 | 5.57 | 21.22 | 31.46 | 18.45 | 12.98 | 10.35 | 7.26 | 5.45 | 3.31 | 4.67 | 5.57 | 21.22 | 31.46 | 18.45 | 12.98 | 10.35 | 4.63 | 4.67 | 5.57 |

* The blood plasma concentrations at t = 0 to t = 12 are the mean blood plasma concentrations observed on day 7 with a 4 mg dose of silodosin once daily for 7 days and are taken from Table 1a. The blood plasma concentrations at t = 12.25 to t = 24 are estimated based upon the values for t = 0 to t = 12, assuming no dose accumulation.

TABLE 2a
Blood Plasma Concentrations (ng/ml) Observed on Day 1 and
Day 7 with a 4 mg Dose of Silodosin Once Daily for 7 Days

| | Time (hours) | | | | | | | | | | | | | |
|---|---|---|---|---|---|---|---|---|---|---|---|---|---|---|
| Subject | 0.0 | 0.3 | 0.5 | 1.0 | 1.5 | 2.0 | 3.0 | 4.0 | 6.0 | 8.0 | 12.0 | 16.0 | 24.0 | 48.0 |
| 1 | 0.00 | 0.00 | 0.00 | 0.52 | 3.65 | 11.16 | 20.28 | 15.53 | 6.74 | 6.13 | 3.57 | 2.87 | 1.83 | 1.46 |
| 2 | 0.00 | 0.00 | 0.59 | 3.16 | 6.54 | 31.67 | 35.31 | 20.33 | 5.45 | 3.83 | 1.81 | 1.26 | 1.09 | 1.31 |
| 3 | 0.00 | 0.00 | 0.00 | 2.09 | 21.09 | 39.89 | 21.82 | 20.90 | 7.15 | 4.83 | 2.41 | 1.53 | 1.04 | 1.44 |
| 4 | 0.00 | 0.00 | 0.00 | 23.99 | 27.06 | 20.22 | 12.50 | 9.70 | 4.71 | 4.25 | 2.80 | 2.54 | 1.42 | 2.05 |
| 5 | 0.00 | 0.00 | 1.48 | 12.07 | 12.49 | 12.40 | 11.54 | 9.88 | 5.52 | 5.63 | 3.23 | 2.46 | 1.98 | 2.52 |
| 6 | 0.00 | 0.00 | 1.43 | 15.32 | 31.22 | 39.39 | 35.57 | 26.51 | 10.22 | 6.81 | 4.00 | 3.12 | 3.01 | 4.68 |
| 7 | 0.00 | 0.00 | 0.51 | 5.75 | 11.36 | 13.54 | 13.29 | 12.01 | 6.99 | 5.05 | 2.70 | 1.84 | 2.04 | 2.32 |
| 8 | 0.00 | 0.00 | 5.48 | 21.73 | 33.78 | 40.29 | 30.14 | 17.04 | 6.78 | 6.63 | 4.93 | 3.64 | 3.07 | 4.43 |
| 9 | 0.00 | 0.00 | 0.00 | 3.77 | 14.50 | 16.22 | 19.72 | 15.06 | 6.49 | 5.83 | 3.50 | 2.62 | 1.59 | 1.90 |
| mean | 0.00 | 0.00 | 1.05 | 9.82 | 17.97 | 24.98 | 22.24 | 16.33 | 6.67 | 5.44 | 3.22 | 2.43 | 1.90 | 2.46 |
| std. dev. | 0.00 | 0.00 | 1.76 | 8.82 | 10.84 | 12.69 | 9.43 | 5.56 | 1.57 | 1.03 | 0.92 | 0.77 | 0.74 | 1.26 |
| | Time (hours) | | | | | | | | | | | | | |
| Subject | 72.0 | 96.0 | 120.0 | 144.0 | 144.3 | 144.5 | 145.0 | 145.5 | 146.0 | 147.0 | 148.0 | 150.0 | 152.0 | 156.0 |
| 1 | 1.60 | 1.61 | 2.12 | 1.05 | 1.04 | 0.97 | 4.94 | 11.96 | 15.50 | 18.27 | 15.60 | 7.06 | 5.75 | 3.27 |
| 2 | 1.01 | 0.84 | 1.05 | 1.03 | 1.27 | 1.74 | 6.64 | 18.00 | 30.03 | 32.65 | 15.37 | 6.54 | 4.22 | 2.01 |
| 3 | 0.90 | 1.56 | 1.64 | 1.84 | 2.01 | 2.01 | 2.82 | 15.00 | 69.31 | 71.33 | 38.58 | 8.23 | 5.51 | 2.60 |
| 4 | 0.67 | 0.91 | 0.00 | 1.53 | 1.15 | 2.41 | 19.16 | 28.69 | 23.50 | 12.94 | 8.07 | 5.23 | 4.33 | 2.44 |
| 5 | 1.75 | 1.37 | 2.73 | 0.99 | 1.28 | 2.44 | 8.61 | 13.30 | 26.51 | 21.81 | 11.71 | 6.84 | 4.88 | 2.77 |
| 6 | 3.43 | 2.28 | 4.09 | 3.79 | 4.21 | 4.55 | 12.10 | 26.92 | 47.01 | 63.28 | 37.69 | 14.41 | 10.17 | 6.17 |
| 7 | 1.99 | 1.41 | 2.01 | 2.12 | 2.06 | 5.94 | 17.17 | 15.97 | 18.75 | 20.07 | 19.03 | 8.27 | 5.60 | 3.11 |
| 8 | 2.21 | 2.65 | 3.22 | 2.58 | 2.65 | 4.65 | 15.90 | 41.93 | 43.97 | 21.68 | 11.61 | 7.33 | 7.43 | 4.49 |
| 9 | 1.07 | 1.16 | 1.78 | 0.63 | 0.57 | 0.55 | 0.57 | 2.02 | 8.78 | 30.01 | 18.36 | 7.06 | 5.50 | 2.92 |
| mean | 1.63 | 1.53 | 2.07 | 1.73 | 1.80 | 2.81 | 9.77 | 19.31 | 31.48 | 32.45 | 19.56 | 7.89 | 5.93 | 3.31 |
| std. dev. | 0.86 | 0.60 | 1.20 | 0.99 | 1.10 | 1.83 | 6.65 | 11.61 | 18.88 | 20.71 | 11.08 | 2.61 | 1.85 | 1.28 |

TABLE 2b
Blood Plasma Concentrations (ng/ml) Observed on Day 1 and Day 7
with a 8 mg Dose of Silodosin Once Daily for 7 Days

| | Time (hours) | | | | | | | | | | | | | |
|---|---|---|---|---|---|---|---|---|---|---|---|---|---|---|
| Subject | 0.0 | 0.3 | 0.5 | 1.0 | 1.5 | 2.0 | 3.0 | 4.0 | 6.0 | 8.0 | 12.0 | 16.0 | 24.0 | 48.0 |
| 1 | 0.00 | 1.47 | 13.85 | 42.13 | 44.81 | 32.81 | 16.72 | 11.26 | 5.48 | 3.51 | 1.60 | 0.95 | 0.56 | 1.07 |
| 2 | 0.00 | 0.00 | 0.00 | 9.29 | 44.23 | 54.51 | 63.97 | 47.45 | 36.53 | 24.21 | 13.24 | 9.18 | 6.96 | 8.23 |
| 3 | 0.00 | 2.27 | 28.23 | 65.47 | 64.19 | 54.56 | 25.30 | 17.15 | 9.28 | 7.74 | 3.94 | 2.79 | 1.27 | 1.72 |
| 4 | 0.00 | 0.00 | 8.54 | 19.61 | 32.61 | 37.94 | 62.68 | 15.01 | 5.62 | 4.39 | 2.67 | 1.66 | 1.72 | 2.20 |
| 5 | 0.00 | 0.00 | 5.00 | 43.31 | 70.27 | 59.01 | 26.08 | 13.52 | 7.97 | 6.60 | 4.38 | 2.94 | 2.80 | 3.37 |
| 6 | 0.00 | 0.00 | 5.01 | 57.51 | 44.48 | 28.65 | 23.58 | 17.81 | 9.94 | 6.78 | 5.42 | 3.19 | 2.39 | 3.35 |
| 7 | 0.00 | 0.00 | 6.16 | 55.35 | 65.65 | 66.47 | 57.85 | 40.28 | 21.95 | 16.78 | 8.57 | 5.41 | 4.27 | 2.67 |
| 8 | 0.00 | 0.00 | 1.71 | 32.22 | 60.05 | 73.38 | 58.16 | 50.39 | 25.09 | 20.37 | 16.07 | 12.17 | 9.38 | 12.14 |
| 9 | 0.00 | 0.00 | 51.16 | 83.89 | 101.60 | 83.90 | 50.53 | 29.05 | 15.44 | 9.54 | 4.83 | 4.20 | 3.48 | 2.71 |
| mean | 0.00 | 0.42 | 13.30 | 45.42 | 58.65 | 54.58 | 42.76 | 26.88 | 15.26 | 11.10 | 6.75 | 4.72 | 3.65 | 4.16 |
| std. dev. | 0.00 | 0.85 | 16.50 | 23.15 | 20.43 | 18.70 | 19.37 | 15.41 | 10.60 | 7.46 | 4.93 | 3.69 | 2.86 | 3.63 |
| Subject | Time (hours) | | | | | | | | | | | | | |
| 1 | 72.0 | 96.0 | 120.0 | 144.0 | 144.3 | 144.5 | 145.0 | 145.5 | 146.0 | 147.0 | 148.0 | 150.0 | 152.0 | 156.0 |
| 2 | 3.12 | 3.55 | 1.75 | 1.95 | 1.60 | 11.63 | 41.51 | 65.51 | 54.34 | 52.40 | 30.19 | 11.08 | 8.40 | 5.04 |
| 3 | 5.69 | 8.26 | 8.32 | 6.99 | 9.65 | 14.52 | 24.58 | 32.58 | 45.40 | 51.86 | 35.37 | 18.88 | 15.18 | 9.45 |
| 4 | 2.58 | 2.86 | 2.44 | 2.40 | 2.44 | 25.23 | 72.25 | 63.27 | 56.03 | 33.38 | 15.51 | 9.47 | 7.79 | 3.86 |
| 5 | 3.03 | 1.92 | 2.09 | 1.90 | 2.00 | 2.01 | 12.58 | 35.49 | 42.13 | 35.86 | 17.49 | 10.20 | 9.68 | 4.49 |
| 6 | 3.72 | 3.32 | 3.42 | 4.15 | 9.25 | 40.90 | 46.27 | 39.01 | 30.68 | 17.49 | 10.60 | 8.31 | 7.43 | 4.80 |
| 7 | 3.84 | 3.43 | 4.47 | 3.94 | 23.98 | 21.98 | 62.96 | 55.57 | 62.78 | 47.15 | 30.50 | 11.39 | 9.77 | 8.22 |
| 8 | 3.39 | 3.78 | 4.71 | 4.87 | 3.64 | 5.69 | 26.49 | 63.87 | 62.92 | 53.17 | 36.46 | 19.22 | 14.50 | 7.91 |
| 9 | 11.41 | 11.18 | 12.89 | 15.77 | 13.11 | 16.93 | 113.90 | 129.20 | 106.10 | 88.84 | 72.43 | 57.30 | 42.35 | 23.11 |
| 10 | 7.41 | 4.50 | 6.05 | 8.04 | 7.98 | 56.28 | 103.20 | 127.00 | 113.80 | 61.25 | 50.08 | 20.36 | 16.57 | 16.51 |
| mean | 4.91 | 4.76 | 5.13 | 5.56 | 8.18 | 21.69 | 55.97 | 67.94 | 63.80 | 49.04 | 33.18 | 18.47 | 14.63 | 9.27 |
| std. dev. | 2.88 | 2.99 | 3.58 | 4.39 | 7.18 | 17.31 | 35.25 | 36.36 | 28.16 | 19.96 | 19.12 | 15.29 | 10.94 | 6.49 |

TABLE 2c
Estimated Blood Plasma Concentrations (ng/ml) on Day 7 with
a 4 mg Dose of Silodosin Twice Daily for 7 Days
Time (hours)

| 0 (t₀) | 0.25 | 0.5 | 1 | 1.5 | 2 | 3 | 4 | 6 | 8 | 12 | 12.25 | 12.5 | 13 | 13.5 | 14 | 15 | 16 | 18 | 20 | 24 |
|---|---|---|---|---|---|---|---|---|---|---|---|---|---|---|---|---|---|---|---|---|
| 1.73 | 1.80 | 2.81 | 9.77 | 19.31 | 31.48 | 32.45 | 19.56 | 7.89 | 5.93 | 1.73 | 1.80 | 2.81 | 9.77 | 19.31 | 31.48 | 32.45 | 19.56 | 7.89 | 5.93 | 3.31 |

* The blood plasma concentrations at t = 0 to t = 8 are the mean blood plasma concentrations observed on day 7 with a 4 mg dose of silodosin once daily for 7 days and are taken from Table 2a. The blood plasma concentrations at t = 12 to t = 24 are estimated based upon the values for t = 0 to t = 12, assuming no dose accumulation.

This is in contrast to administration of a divided daily dose (i.e., twice a day), which exhibits Cₘₐₓ at about 2 hours and about 14 hours after the first administration of the day.

As illustrated in Tables 1 and 2 and FIGS. 1 and 2, the Cₘₐₓ for a 4 mg dose of silodosin is typically about half the Cₘₐₓ for an 8 mg dose of silodosin. About 12 hours after administration, the blood plasma concentration falls to near baseline. Preferably, “near baseline” blood plasma concentration of silodosin is about 25%, or less, of Cₘₐₓ at steady state, more preferably about 20%, or less, of Cₘₐₓ at steady state, and most preferably about 15%, or less, of Cₘₐₓ at steady state.

As illustrated in Tables 6 and 7 below, administering silodosin as a single 8 mg dose once daily produces mean changes in IPSS/AUASS and Qₘₐₓ that are comparable to those for a divided daily dose of 4 mg twice daily. Thus, efficacy in treating the symptoms associated with BPH is unexpectedly provided with the 24 hour dosing regimen, even though blood plasma concentrations of silodosin are substantively reduced over the dosing interval and near baseline by approximately 12 hours.

Further, as illustrated in Table 8 below, administering silodosin as a single 8 mg dose once daily does not produce a concomitant increase in cardiovascular adverse events (such as orthostatic hypotension) relative to placebo controls, as compared to those exhibited when administering a 4 mg dose twice daily, despite a two-fold increase in Cₘₐₓ. This is unexpected because the incidence of adverse events is often directly proportional to maximum blood concentration.

The pharmaceutical composition of silodosin typically comprises silodosin or a pharmaceutically acceptable salt thereof and at least one pharmaceutically acceptable excipient. Pharmaceutically acceptable excipients include, for example, fillers, diluents, disintegrants, glidants, lubricants, and other excipients known in the art. In one preferred embodiment, the pharmaceutical composition of silodosin comprises silodosin, mannitol, pregelatinized starch, sodium lauryl sulfate, and magnesium stearate. Compositions useful in the methods of the invention are described, for example, in U.S. Publication No. 2006/0018959, which is incorporated herein by reference in its entirety.

While solid dosage forms are preferred, the once-daily dosage regimens of the invention are meant to include any dosage form, including liquid (e.g., a syrup) and semi-solid (e.g., a gel) dosage forms.

The pharmaceutical composition may be formulated into a solid dosage form by any method known to a person of ordinary skill in the art. Such methods include, but are not limited to, wet granulation, dry granulation by slugging and/or roller compaction, and direct compression. The solid dosage form may be in the form of a tablet (e.g., a compressed dosage form) or in the form of a capsule containing silodosin, optionally with one or more pharmaceutically acceptable excipients. The silodosin may be granulated, for example, with the pharmaceutically acceptable excipients.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Pharmacokinetic Study on Silodosin (Study US011)

A double-blind, placebo-controlled, multiple-dose, parallel pharmacokinetic investigation was conducted in 36 healthy male subjects, 30-70 years of age, in a fed state. Nine subjects in each of the three dosing cohorts of 12 subjects received 4, 8, or 12 mg of silodosin once daily for seven days. The remaining three subjects in each dosing cohort received placebo. Mean pharmacokinetic parameters (with standard deviation) on days 1 and 7 for dose cohorts 4 and 8 mg are described in Table 3 below.

TABLE 3
Pharmacokinetics of Silodosin

| Dose of Silodosin | Day | Cₘₐₓ (ng/ml) | Mean Tₘₐₓ (h) | AUC (ng * h/ml) | t½ (h) |
|---|---|---|---|---|---|
| 4 mg | 1 | 27.6 (11.5)* | 2.0 | 169.0 (48.4)* | 10.7 (2.04)* |
| 8 mg | 1 | 63.2 (19.3) | 1.5 | 413.0 (202) | 11.2 (6.93) |
| 4 mg | 7 | 37.2 (18.7) | 3.0 | 171 (67.1) | 12.5 (5.89) |
| 8 mg | 7 | 73.4 (32.4) | 1.5 | 396 (221) | 10.6 (4.55) |

*Unless otherwise specifically noted, numbers in parenthesis “( )” in this and the other tables herein denotes the standard deviation.

As illustrated in Table 3, a doubling of the silodosin dose caused an approximately two-fold increase in Cₘₐₓ and AUC.

### Example 2

### Pharmacokinetic Study on Silodosin (Study 98364)

A double-blind, placebo-controlled, multiple-dose, parallel pharmacokinetic investigation was conducted in 24 healthy male subjects, 30-70 years of age. Six subjects in each of the three dosing cohorts of eight subjects received 4, 8, or 12 mg of silodosin for seven days (once daily on Day 1, twice daily on Days 2-6, and once daily on Day 7). The remaining two subjects in each dosing cohort received placebo.

One subject was discontinued in 4 mg treatment, so data from only five subjects is presented in Table 1a above.

### Example 3

### Efficacy Studies on Silodosin

Study A: 4 mg Silodosin Twice Daily (KMD-202)—Japanese Phase 2 Study

A multi-center, randomized, double-blind, placebo-controlled parallel, 4-week treatment phase clinical investigation in 271 Japanese patients each receiving 2 or 4 mg silodosin or placebo twice daily. The primary objective was to test the hypothesis that the effectiveness of silodosin was superior to placebo for the relief of subjective and objective symptoms of BPH as measured by IPSS/AUASS, Qₘₐₓ, and a quality of life (“QoL”) question (i.e., question 8 of the IPSS/AUASS: “if you were to spend the rest of your life with your urinary condition the way it is now, how would you feel about that?”) Safety was assessed on a four-category scale according to the presence/absence, type, and severity of adverse events and laboratory test values. Patients were males who were at least 50 years of age and who had at least two of the following three variables classified as moderate, or at least one variable classified as severe: IPSS/AUASS, Qₘₐₓ and QoL.

Study B: 4 mg Silodosin Twice Daily (KMD-303)—Japanese Phase 3 Study

A multi-center, randomized, double-blind, placebo-controlled parallel, 12-week treatment phase clinical investigation in 457 Japanese patients each receiving 4 mg silodosin or placebo twice daily, or tamsulosin 0.2 mg once daily was conducted. The primary objective was to test the hypothesis that the effectiveness of silodosin was superior to placebo for the relief of subjective and objective symptoms of BPH as measured by IPSS/AUASS and Qₘₐₓ. Safety was assessed by monitoring adverse events, clinical laboratory measurements, vital signs, and physical exams. Patients were males who were at least 50 years of age and who had a total IPSS/AUASS of greater than or equal to 8, a QoL of greater than or equal to 8, a Qₘₐₓ of less than 15 ml/sec, and a post-void bladder residual volume of less than 100 ml.

Study C: 4 mg or 8 mg Silodosin Once Daily (KMD-3213-US021-99)—U.S. Phase 2 Study

A multi-center, randomized, double-blind, placebo-controlled, parallel, 8-week treatment phase clinical investigation in 264 patients each receiving silodosin 4 or 8 mg once daily, or placebo once daily was conducted. The 6-week stable dosing period was preceded by a 4-week placebo lead-in and a 2-week titration period. The primary objectives were to test the hypothesis that the effectiveness of silodosin was superior to placebo for the relief of symptoms of BPH as measured by a baseline to endpoint change in the total score of the IPSS/AUASS and maximum urine flow rate, and to compare the safety of silodosin to placebo using an evaluation of adverse events, vital signs, ECGs, clinical laboratory tests, and physical exams. Male patients were selected who were at least 45 years of age with signs and symptoms of BPH (AUASS of ≧13 and maximum urine flow rate between 4 and 15 mL/sec).

The results for IPSS/AUASS for study C are shown in Table 4 and the results for Qₘₐₓ for study C are shown in Table 5 below.

TABLE 4
Total IPSS/AUASS Score for Study C.

| | | | Visit 3 | Visit 4 | Visit 5 | Visit 6 | Visit 7 |
|---|---|---|---|---|---|---|---|
| | | | (baseline, Day 29) | (Day 36) | (Day 43) | (Day 64) | (EOS, Day 84) |
| Modified Intent to Treat | 8 mg silodosin | N | 90 | 90 | 90 | 90 | 90 |
| (LOCF) | | Mean (SD) | 20.8 (5.3) | 18.0 (6.0) | 16.5 (6.0) | 15.9 (6.4) | 14.0 (6.5) |
| | | Mean change (SD) | | −2.7 (3.7) | −4.3 (3.9) | −4.9 (4.6) | −6.8 (5.8) |
| | 4 mg silodosin | N | 88 | 88 | 88 | 88 | 88 |
| | | Mean (SD) | 19.7 (5.1) | 17.3 (5.6) | 15.5 (5.6) | 14.6 (6.0) | 13.9 (6.0) |
| | | Mean change (SD) | | −2.4 (4.0) | −4.2 (4.5) | −5.1 (5.1) | −5.7 (5.5) |
| | Placebo | N | 83 | 83 | 83 | 83 | 83 |
| | | Mean (SD) | 19.7 (5.2) | 17.0 (5.4) | 16.9 (6.2) | 16.2 (6.1) | 15.7 (6.7) |
| | | Mean change (SD) | | −2.7 (3.8) | −2.8 (4.2) | −3.5 (5.2) | −4.0 (5.5) |
| | P-Values | Overall | 0.2895 | 0.8070 | 0.0521 | 0.0638 | 0.0064 |
| | | 8 mg vs. placebo | 0.3269 | 0.7065 | 0.0335 | 0.0957 | 0.0018 |
| | | 4 mg vs. placebo | 0.5850 | 0.5141 | 0.0350 | 0.0230 | 0.0355 |
| | | 8 mg vs. 4 mg | 0.1204 | 0.7794 | 0.9849 | 0.5331 | 0.2871 |

* LOCF = Last observation carried forward;
SD = standard deviation

TABLE 5
Qₘₐₓ for Study C.

| | | | Visit 3 | | Visit 4 | Visit 5 | Visit 6 | Visit 7 |
|---|---|---|---|---|---|---|---|---|
| | | | (baseline, Day 29) | | (Day 36) | (Day 43) | (Day 64) | (EOS, Day 84) |
| | | | Pre-dose | Post-dose | Post-dose | Post-dose | Post-dose | Post-dose |
| Q-max (ml/sec) | | | (—) | (2-6 hr) | (2-6 hr) | (2-6 hr) | (2-6 hr) | (2-6 hr) |
| Modified Intent to Treat | 8 mg silodosin | N | 90 | 90 | 90 | 90 | 90 | 90 |
| (LOCF) | | Mean (SD) | 9.6 (2.7) | 11.7 (3.5) | 12.5 (4.8) | 12.5 (5.7) | 12.9 (5.6) | 13.0 (5.8) |
| | | Mean change (SD) | | 2.0 (3.1) | 2.9 (4.4) | 2.9 (5.4) | 3.2 (5.5) | 3.5 (5.7) |
| | 4 mg silodosin | N | 88 | 88 | 88 | 88 | 88 | 88 |
| | | Mean (SD) | 9.7 (3.0) | 12.2 (4.4) | 12.4 (4.7) | 12.2 (4.7) | 12.0 (4.3) | 12.6 (4.4) |
| | | Mean change (SD) | | 2.5 (3.6) | 2.7 (4.4) | 2.6 (4.2) | 2.3 (4.1) | 2.9 (4.0) |
| | Placebo | N | 83 | 83 | 83 | 83 | 83 | 83 |
| | | Mean (SD) | 10.1 (2.7) | 11.5 (3.9) | 11.2 (3.8) | 11.1 (3.8) | 11.7 (4.9) | 11.6 (4.8) |
| | | Mean change (SD) | | 1.4 (3.7) | 1.0 (3.7) | 1.0 (3.8) | 1.6 (4.7) | 1.5 (4.4) |
| | P-values | Overall | 0.5813 | 0.1371 | 0.0257 | 0.0461 | 0.1540 | 0.0521 |
| | | 8 mg vs. placebo | 0.4082 | 0.4148 | 0.0163 | 0.0218 | 0.0550 | 0.0174 |
| | | 4 mg vs. placebo | 0.3324 | 0.0482 | 0.0204 | 0.0454 | 0.4205 | 0.0966 |
| | | 8 mg vs. 4 mg | 0.8837 | 0.2330 | 0.9336 | 0.7633 | 0.2542 | 0.4601 |

* LOCF = Last observation carried forward;
SD = standard deviation;
LOCF was performed on 2-6 hour data only

Study D: 8 mg Silodosin Once Daily (SI04009)—U.S. Phase 3 Study

A pivotal, multi-center, randomized, double-blind, placebo-controlled parallel, 4-week placebo lead-in and 12-week treatment phase clinical investigation in 461 patients receiving 8 mg silodosin or placebo once daily was conducted. The primary objective was to test the hypothesis that the effectiveness of silodosin was superior to placebo for the relief of symptoms of BPH as measured by a baseline to endpoint change in the total score of the IPSS/AUASS. Secondary objectives were to test the hypothesis that the effectiveness of silodosin was superior to placebo in a baseline to endpoint change in the maximum urine flow rate (Qₘₐₓ), and to compare the safety of silodosin to placebo using an evaluation of adverse events, vital signs, ECGs, clinical laboratory tests, and physical exams. Patients were males who were at least 50 years of age and who had signs and symptoms of BPH, i.e., an IPSS/AUASS of greater than or equal to 13, a Qₘₐₓ of between 4 and 15 ml/sec, and a post-void bladder residual volume of less than 250 ml.

Study E: 8 mg Silodosin Once Daily (SI04010)—U.S. Phase 3 Study

A pivotal, multi-center, randomized, double-blind, placebo-controlled parallel, 4-week placebo lead-in and 12-week treatment phase clinical investigation in 462 patients receiving 8 mg silodosin or placebo once daily (same design as SI04009) was conducted. The primary objective was to test the hypothesis that the effectiveness of silodosin was superior to placebo for the relief of symptoms of BPH as measured by a baseline to endpoint change in the total score of the IPSS/AUASS. Secondary objectives were to test the hypothesis that the effectiveness of silodosin was superior to placebo in a baseline to endpoint change in the maximum urine flow rate, and to compare the safety of silodosin to placebo using an evaluation of adverse events, vital signs, ECGs, clinical laboratory tests, and physical exams. Patients were males who were at least 50 years of age (between 45 and 75 years of age) and who had signs and symptoms of BPH, i.e., an IPSS/AUASS of greater than or equal to 13, a Qₘₐₓ of between 4 and 15 ml/sec, and a post-void bladder residual volume of less than 250 ml.

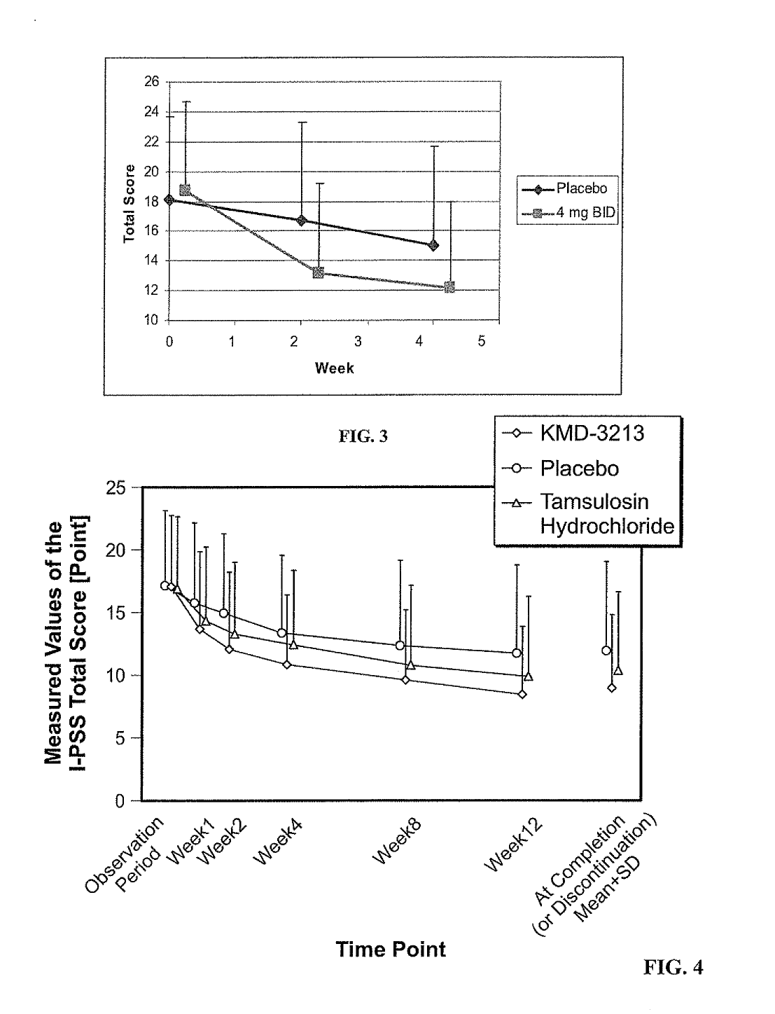

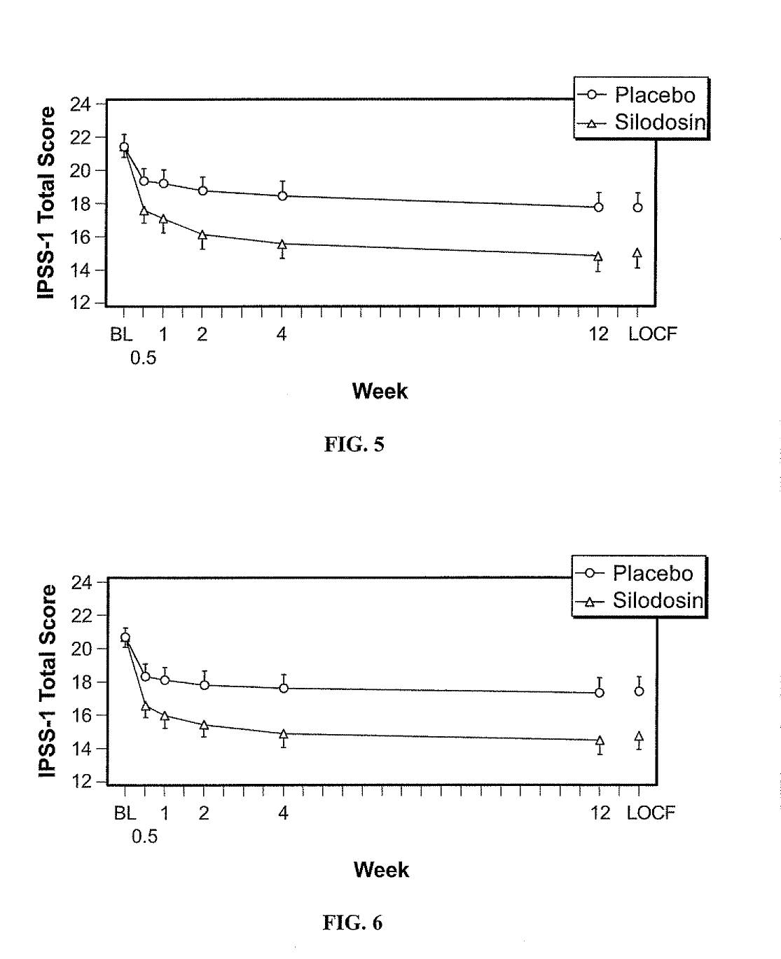

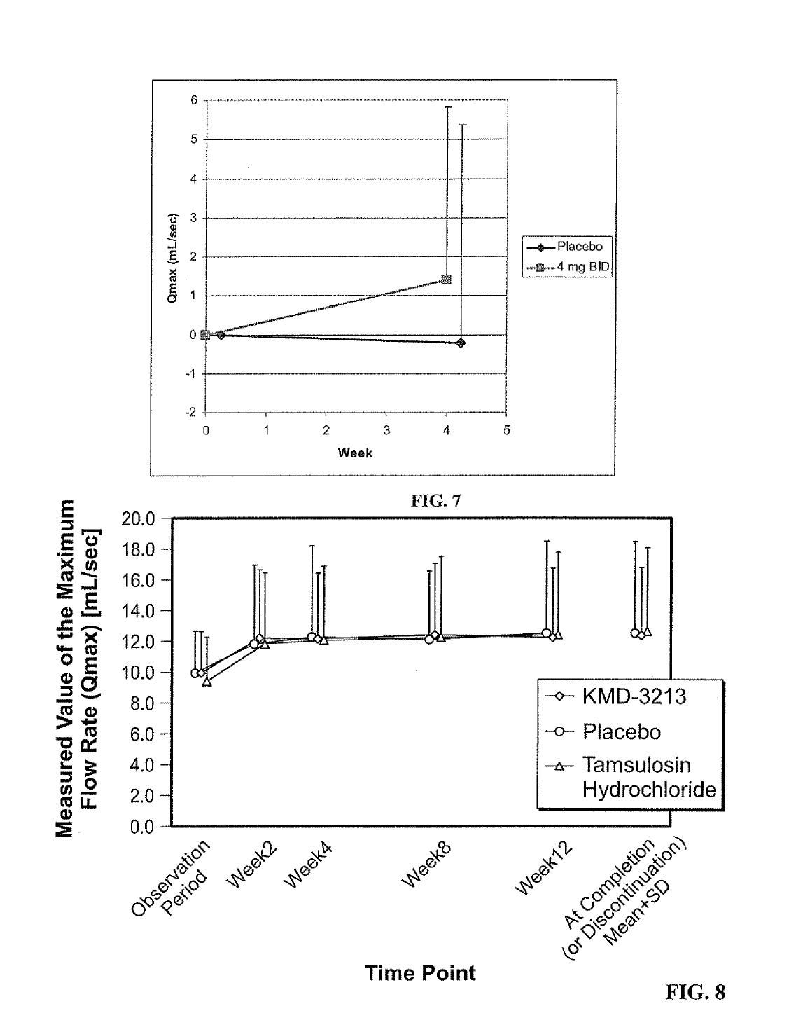

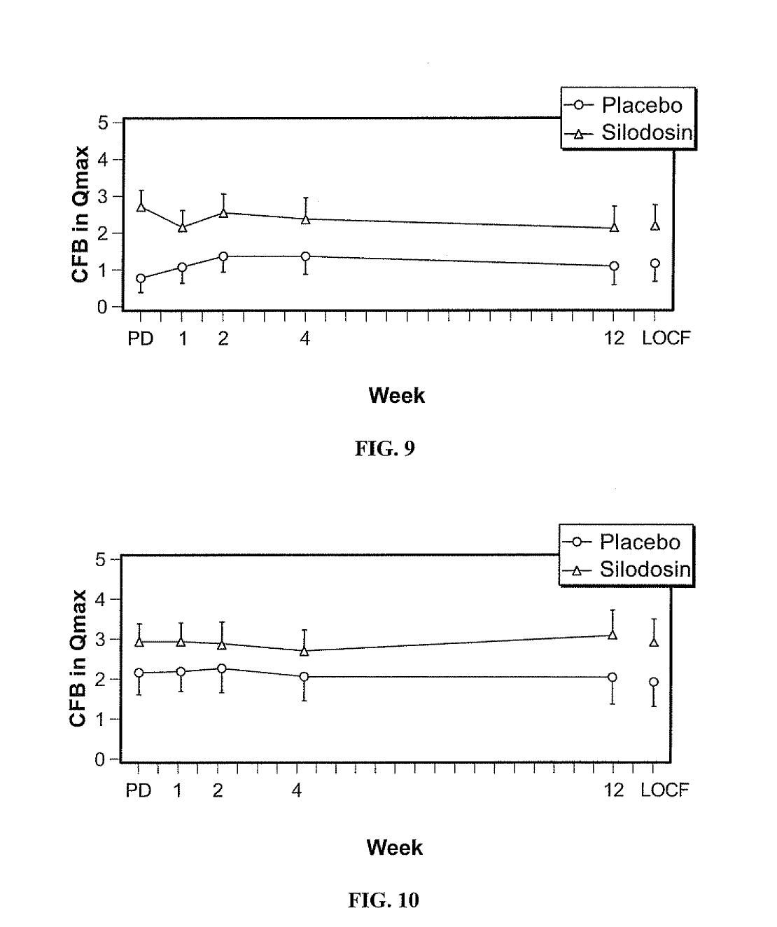

IPSS/AUASS, Qₘₐₓ, and adverse events were measured in studies A-E and the results are summarized in Tables 6-8 below. In addition, the results for IPSS/AUASS for studies A, B, D and E are illustrated in FIGS. 3 to 6, respectively, and the results for Qₘₐₓ for studies A, B, D and E are illustrated in FIGS. 7 to 10, respectively.

TABLE 6
Mean Change From Baseline (CFB) in IPSS/AUASS
during Silodosin and Placebo Therapy

| | | Week | | | | | | | |
|---|---|---|---|---|---|---|---|---|---|
| | | 0 (baseline | 0.5 | 1 | 2 | 4 | 5 | 8 | 12 |
| Study | Dose | value) | (CFB) | (CFB) | (CFB) | (CFB) | (CFB) | (CFB) | (CFB) |
| A | Placebo | 18.1 | | | −1.6 | −3.0 | | | |
| | 4 mg (twice | 18.7 | | | −5.6 | −6.6 | | | |
| | daily) | | | | | | | | |
| B | Placebo | 17.1 | | −1.2 | −2.2 | −3.8 | | −4.6 | −5.3 |
| | 4 mg (twice | 17.1 | | −3.4 | −4.9 | −6.3 | | −7.7 | −8.9 |
| | daily) | | | | | | | | |
| C | Placebo | 19.7 | | −2.7 | −2.8 | | −3.5 | −4.0 | |
| | 4 mg (once | 19.7 | | −2.4 | −4.2 | | −5.1 | −5.7 | |
| | daily) | | | | | | | | |
| | 8 mg (once | 20.8 | | −2.7 | −4.3 | | −4.9 | −6.8 | |
| | daily) | | | | | | | | |
| D | Placebo | 21.4 | −2.0 | −2.1 | −2.5 | −2.9 | | | −3.7 |
| | 8 mg (once | 21.5 | −3.9 | −4.5 | −5.3 | −5.9 | | | −6.7 |
| | daily) | | | | | | | | |
| E | Placebo | 21.2 | −2.5 | −2.8 | −3.1 | −3.4 | | | −3.6 |
| | 8 mg (once | 21.2 | −4.4 | −5.0 | −5.6 | −6.2 | | | −6.6 |
| | daily) | | | | | | | | |

TABLE 7
Mean Change From Baseline (CFB) in Maximum Flow
Rate (ml/sec) during Silodosin and Placebo Therapy

| | | Week | | | | | | | |
|---|---|---|---|---|---|---|---|---|---|
| | | | 2-6 hrs | | | | | | |
| | | | after | | | | | | |
| | | 0 | 1^{st} | | | | | | |
| | | (baseline | dose | 1 | 2 | 4 | 5 | 8 | 12 |
| Study | Dose | value) | (CFB) | (CFB) | (CFB) | (CFB) | (CFB) | (CFB) | (CFB) |
| A | Placebo | 10.63 | | | | −0.17 | | | |
| | 4 mg | 10.70 | | | | 1.31 | | | |
| | (twice | | | | | | | | |
| | daily) | | | | | | | | |
| B | Placebo | 9.93 | | | 1.74 | 2.19 | | 1.99 | 2.35 |
| | 4 mg | 9.89 | | | 2.23 | 2.18 | | 2.38 | 2.33 |
| | (twice | | | | | | | | |
| | daily) | | | | | | | | |
| C | Placebo | 10.1 | 1.4 | 1.0 | 1.0 | | 1.6 | 1.5 | |
| | 4 mg | 9.7 | 2.5 | 2.7 | 2.6 | | 2.3 | 2.9 | |
| | (once | | | | | | | | |
| | daily) | | | | | | | | |
| | 8 mg | 9.6 | 2.0 | 2.9 | 2.9 | | 3.2 | 3.4 | |
| | (once | | | | | | | | |
| | daily) | | | | | | | | |
| D | Placebo | 9.0 | 0.8 | 1.1 | 1.4 | 1.4 | | | 1.1 |
| | 8 mg | 9.0 | 2.7 | 2.2 | 2.6 | 2.4 | | | 2.1 |
| | (once | | | | | | | | |
| | daily) | | | | | | | | |
| E | Placebo | 8.7 | 2.1 | 2.2 | 2.2 | 2.0 | | | 2.0 |
| | 8 mg | 8.4 | 2.9 | 2.9 | 2.9 | 2.7 | | | 3.1 |
| | (once | | | | | | | | |
| | daily) | | | | | | | | |

TABLE 8
Incidence of Adverse Events (% of patients) during
Silodosin and Placebo Therapy

| | | | Orthostatic | |
|---|---|---|---|---|
| Study | Dose | Dizziness | Hypotension | Syncope |
| A | Placebo | 2.2 | 0 | 0 |
| | 4 mg | 2.2 | 1.1 | 0 |
| | (twice daily) | | | |
| B | Placebo | 2.2 | 0 | 0 |
| | 4 mg | 2.3 | 3.4 | 0 |
| | (twice daily) | | | |
| C | Placebo | 7.0 | 2.3 | 0 |
| | 4 mg | 9.1 | 4.5 | 0 |
| | (once daily) | | | |
| | 8 mg | 5.6 | 3.3 | 0 |
| | (once daily) | | | |
| D | Placebo | 1.8 | 2.2 | 0 |
| | 8 mg | 2.6 | 2.6 | 0 |
| | (once daily) | | | |
| E | Placebo | 0.4 | 0.9 | 0 |
| | 8 mg | 3.9 | 2.6 | 0.4¹ |
| | (once daily) | | | |

¹Only one subject exhibited syncope, and that subject was using an excluded medication (Prazosin) during the study period. It is believed that the syncope was due to the use of this excluded medication.

The data in Tables 6-8 illustrate that administering silodosin as a single dose 8 mg once daily instead of a twice daily dose of 4 mg per dose produces comparable treatment effects on IPSS/AUASS and Qₘₐₓ without conferring additional safety risks, i.e., with a concomitant increase in side effects relative to a twice daily administration of 4 mg per dose.

### Example 4

### Method of Preparing Silodosin Capsules

Silodosin capsules were produced from the ingredients listed in Table 9 below, by the following method.

TABLE 9
Silodosin Capsule Formulation

| Ingredient | Unit amount (mg) | Batch amount (kg) |
|---|---|---|
| Silodosin | 8 | 3.7 |
| Mannitol, USP (Pearlitol 50C) | 264.8 | 122.5 |
| Pregelatinized starch, NF | 52 | 24.1 |
| (PCS PC-10) | | |
| Pregelatinized starch, NF | 18 | 8.3 |
| Purified water, USP | 0 | 32 |
| Sodium lauryl sulfate, NF | 3.6 | 1.7 |
| Magnesium stearate, NF | 3.6 | 1.7 |

Silodosin, mannitol, and starch were granulated in the presence of water using a high-speed granulator. The resulting granulate was then dried in a fluid bed dryer. The dried granulate was then passed through a mill. The lubricants sodium lauryl sulfate and magnesium stearate were combined with the milled granulate and the combination was mixed in a mixer. The granulate was then filled into capsules.

We claim:

1. A method for treating a patient having symptoms associated with benign prostatic hyperplasia comprising administering once daily to said patient a pharmaceutical composition comprising an effective amount of silodosin or a pharmaceutically acceptable salt thereof.

2. The method of claim 1, wherein the effective amount is more than about 4 mg.

3. The method of claim 1, wherein the effective amount is about 4 mg to about 8 mg.

4. The method of claim 3, wherein the effective amount is about 4 mg.

5. The method of claim 3, wherein the effective amount is about 8 mg.

6. The method of claim 1, wherein the pharmaceutical composition further comprises a sugar.

7. The method of claim 6, wherein the sugar is mannitol.

8. The method of claim 1, wherein the pharmaceutical composition comprises a dosage form adapted to release about 50% or more of the silodosin from the dosage form one hour from administration of said dosage form.

9. A method for achieving comparable efficacy in treating a patient having symptoms associated with benign prostatic hyperplasia, comprising administering to said patient once daily a pharmaceutical composition comprising twice the dose of silodosin or a pharmaceutically acceptable salt thereof that is administered in each dose in a twice daily regimen, wherein there is no concomitant increase in cardiovascular adverse side effects associated with the twice daily regimen of a silodosin.

10. The method of claim 9, wherein the cardiovascular adverse side effect is orthostatic hypotension.

11. The method of claim 9, wherein about 8 mg of silodosin or a pharmaceutically acceptable salt thereof is administered once daily.

12. The method of claim 9, wherein 4 mg of silodosin or a pharmaceutically acceptable salt thereof is administered once daily.

13. A method for treating a patient having symptoms associated with benign prostatic hyperplasia over a 24-hour period, comprising administering once daily to said patient a pharmaceutical composition comprising an effective amount of silodosin or a pharmaceutically acceptable salt thereof, wherein silodosin plasma levels of about 25%, or less, of Cₘₐₓ at steady state are achieved in said patient at 12 hours after administration.

14. The method of claim 13, wherein the silodosin plasma level at 12 hours is about 20%, or less, of Cₘₐₓ.

15. The method of claim 13, wherein the silodosin plasma level at 12 hours is about 15%, or less, of Cₘₐₓ.

16. The method of claim 13, wherein the effective amount is more than about 4 mg.

17. The method of claim 13, wherein the effective amount is about 4 mg to about 8 mg.

18. The method of claim 17, wherein the effective amount is about 4 mg.

19. The method of claim 17, wherein the effective amount is about 8 mg.

20. The method of claim 13, wherein the pharmaceutical composition further comprises a sugar.

21. The method of claim 19, wherein the sugar is mannitol.

22. The method of claim 13, wherein the pharmaceutical composition comprises a dosage form adapted to release about 50% or more of the silodosin from the dosage form one hour from administration of said dosage form.

23. A pharmaceutical composition comprising about 8 mg of silodosin or a pharmaceutically acceptable salt thereof and at least one pharmaceutically acceptable excipient in a unitary dosage form.

24. The pharmaceutical composition of claim 23 comprising a dosage form adapted to release about 50% or more of the silodosin from the dosage form one hour from administration of said dosage form.

25. The pharmaceutical composition of claim 23, wherein the pharmaceutically acceptable excipient comprises a sugar.

26. The pharmaceutical composition of claim 25, wherein the sugar is mannitol.
